(19) United States
(12) Patent Application Publication
(10) Pub. No.: US 2021/0379010 A1
(43) Pub. Date: Dec. 9, 2021

(54) COMPOSITIONS COMPRISING CANNABINOIDS FOR USE IN THE TREATMENT OF BIOFILM AND CONDITIONS ASSOCIATED WITH MICROBIAL, FUNGAL, BACTERIAL INFECTIONS

(71) Applicant: Yissum Research Development Company of The Hebrew University of Jerusalem Ltd., Jerusalem (IL)

(72) Inventors: Doron STEINBERG, Jerusalem (IL); Raphael MECHOULAM, Jerusalem (IL)

(73) Assignee: Yissum Research Development Company of The Hebrew University of Jerusalem Ltd., Jerusalem (IL)

(21) Appl. No.: 17/288,942

(22) PCT Filed: Oct. 30, 2019
(86) PCT No.: PCT/IL2019/051177

§ 371 Date: Apr. 27, 2021

Related U.S. Application Data
(60) Provisional application No. 62/752,849, filed on Oct. 30, 2018.

(51) Int. Cl. A61K 31/352; A61K 45/06; A61L 2/18

(52) U.S. Cl. CPC A61K 31/352; A61L 2/18; A61K 45/06

(57) ABSTRACT

The invention provides compositions comprising at least one cannabinoid compound, for use in the method of treating and preventing a disease, condition or symptom caused by, or associated with fungi, bacteria and microbes.

### BACKGROUND OF THE INVENTION

In the United States, drug-resistant bacteria are a leading cause of death due to severe infection. In fact, the number of annual deaths due to common drug-resistant bacteria surpasses those due to smoking and tobacco. Staphylococcus aureus bacterial infections are the source of a number of potentially lethal diseases affecting skin, lung, and blood and whose courses and symptoms depend upon the tissue that becomes infected. While skin infections, including sites of surgery, are quite common and sometimes deadly, the most lethal, and for this reason the best known, are pneumonia due to infection of the lungs or severe sepsis (septic shock) due to infection of the blood. Resistance to antibiotics is a cause for major concern for a number of infectious bacterial strains, and chief amongst them is methicillin-resistant Staphylococcus aureus.

Methicillin-resistant Staphylococcus aureus (“MRSA”) strains account for most hospital-acquired and nursing home-acquired infections and they are a leading cause of mortality due to infection. They are also a leading cause of close quarter community-acquired infections impacting children in daycare centers, members of sports teams, military personnel, and prisoners. The instances of serious MRSA infection in the US has mushroomed in the past decade to the point where the rate of invasive MRSA exceeds the combined rate of invasive infections due to pneumococcal disease, meningococcal disease, group A streptococcus, and Haemophilus influenza. While overall incidents of MRSA are relatively low, the risk of death from an MRSA infection is very high, as is the cost associated with treatment.

As the infection rate increases, there have been fewer unique classes of drugs introduced to combat these infections. Given that only two new antibiotic pharmacophores have been introduced into the clinic over the last 30 plus years (Barrett 2003; Pucci 2006) locating structurally and/or mechanistically novel antimicrobial approaches is of considerable interest. This is especially true given that antibiotic resistance is on the rise (Levy 2004) and the fact that large drug companies are increasingly less interested in supporting antimicrobial discovery programs (Projan 2003). Innovative ways to prevent MRSA infections are clearly needed.

Bacteria communicate and coordinate population behavior through the mechanism of quorum sensing (QS), which controls the expression of genes that affect a variety of bacterial processes. QS is based on small signaling molecules, termed autoinducers (AIs), which control factors such as bioluminescence, pigment production, motility and biofilm formation, among many others. The QS, free-living marine bacterium Vibrio harveyi produces and responds to at least three distinct AIs: HAI-1, an acyl homoserine lactone; AI-2, a furanosylborate-diester; and CAI-1, a long-chain amino ketone (Z)-3-aminoundec-2-en-4-one (Ea-C8-CAI-1). AI-2 is referred to “universal autoinducer” as it is found in numerous Gram-positive and Gram-negative bacteria.

Biofilms are the most common environmental conditions of microbes. The biofilms are associated with most diseases and pathogenic situations in human and animals. They are also associated with numerous environmental, industrial problems. A number of reports have shown that microbial cells growing in biofilms are profoundly resistant to many antibiotics. Biofilms play an intrinsic role in protecting bacterial cells from any fluctuations of the environment, including protecting the colonies from any potential antimicrobial agents. It is well studied that the physiological properties of sessile biofilm populations are different from their planktonic counterparts and contribute to their better survival within the infected hosts.

Biofilm-protected bacterial cells present a different mode of growth, pathogenicity and physiology compared to planktonic cells, and the peculiarity of the mode of growth contributes to manifestation of antibiotic resistance. Due to this reason, treatment for biofilm-related infection becomes increasingly challenging, leading eventually to chronic infections. The biofilm forming ability antimicrobial resistance microbes as of methicillin-resistance Staphylococcus aureus (MRSA) represents a major factor for nosocomial infections and treatments for these infections are further complicated by the presence of other virulent factors such as toxin production and host immune evasion ability.

### SUMMARY OF THE INVENTION

Thus, there is a need for a solution to the spread of infectious diseases, including those caused by drug-resistant bacteria, fungi and/or microbial infections, and particularly those capable of forming biofilms.

The present invention thus provides a composition comprising at least one cannabinoid compound, for use in the treatment of a disease, condition or symptom caused by, or associated with fungi.

In a further aspect, the invention provides a composition comprising at least one cannabinoid compound, for use in the treatment of a disease, condition or symptom caused by, or associated with fungal biofilm. In a further aspect, the invention provides a composition comprising at least one cannabinoid compound, for use in the treatment of a disease, condition or symptom caused by, or associated with planktonic fungi.

In yet another aspect, the invention provides a composition comprising at least one cannabinoid compound, for use in the inhibition of the formation and/or growth of fungal biofilm and/or disruption of fungal biofilm.

The present invention further provides a composition comprising at least one cannabinoid compound, for use in the disintegration of biofilm (i.e. destruction of the biofilm formation caused by any microorganism, thereby inhibiting the cause of disease condition or symptom caused by, or associated with such).

The invention further provides a composition comprising at least one cannabinoid compound, for use in the treatment of a disease, condition or symptom caused by, or associated with drug resistant bacteria.

The invention provides a composition comprising at least one cannabinoid compound, for use in the treatment, prevention or inhibition of a disease, condition or symptom caused by, or associated with the formation or growth of at least one of fungi, fungal biofilm and any combinations thereof.

In some embodiments, said fungi is selected from planktonic fungi, fungal biofilm and any combinations thereof. In other embodiments, said treatment further comprises inhibition of the formation of fungal biofilm, inhibition of the growth of fungal biofilm, inhibition of the disruption of fungal biofilm and any combination thereof. In yet further embodiments, said treatment comprises preventing the formation of fungal biofilm on a surface.

When referring to a “cannabinoid compound” it should be understood to encompass any compound that acts on cannabinoid receptors. Such compounds include, but are not limited to endocannabinoids (produced naturally in the body by animals), phytocannabinoids (found in some plants), synthetic and semi-synthetic cannabinoids (manufactured artificially). In some embodiments, said at least one cannabinoid compound is an endo-cannabinoid compound.

In other embodiments, said at least one cannabinoid compound is select from ARAS (arachidonoyl serine), 2AG (2-arachidonoyl glycerol), AEA (arachidonoyl ethanolamide), OEA (oleoyl ethanolamide), OG (oleoyl glycine), OA (oleoyl alanine), HU-210 (1,1-Dimethylheptyl-11-hydroxy-tetrahydrocannabinol), HU-308 ([(1R,2R,5R)-2-[2,6-dimethoxy-4-(2-methyloctan-2-yl)phenyl]-7,7-dimethyl-4-bicyclo[3.1.1]hept-3-enyl]methanol)), PEA (palmitoyl ethanolamide) HU-433 ([(1R,5R)-2-[2,6-dimethoxy-4-(2-methyloctan-2-yl)phenyl]-6,6-dimethyl-4-bicyclo[3.1.1]hept-3-enyl]methanol), AraG (Arachidonoyl glycine), PG (Palmitoyl glycine), AraA (Arachidonoyl alanine), PA (Palmitoyl alanine), PS (Palmitoyl serine), OS (Oleoyl serine), 2-arachidonoyl glyceryl ether, 2-oleoyl glyceryl ether, 2-palmitoyl glyceryl ether and any derivative or combinations thereof.

When referring to “biofilm” it should be understood to encompass a cohort of microorganisms (including aerobic/anaerobic/facultative bacteria, fungi, virus, such as for example: staphylococci, enterococci, actinomyces, micobacteriu, enterobacteriaceae pseudomonadaceae, firmicutes, candida, aspargili micro sporidia, chytridiomycota, blastocladiomycota, neocallimastigomycota, glomeromycota, ascomycota, and basidiomycota and also drug resistant microbes as: MRSA, MRSE, VRE, CRE FRC, and so forth) in which cells stick to each other and often also to a surface (including any type of living or non-living surfaces such as plastic, polymers, artificial devices, implants, indwelling devices, liquid surfaces, air-liquid, submerge biofilm, pellicle, any type of solid surfaces, biological surfaces such as skin, mucosal tissue, bone, teeth, natural or non-natural soft surfaces). These adherent cells become embedded within an extracellular matrix that is composed of extracellular polymeric substances (EPS). The EPS components are produced by the cells within the biofilm and are typically a polymeric conglomeration of extracellular DNA, proteins, and/or polysaccharides. The biofilm formed by these microorganisms has a three-dimensional structure and represent a community for microorganisms and thus the microbial cells growing in a biofilm are physiologically distinct from planktonic cells of the same organism, which, by contrast, are cells that may float or swim in a liquid medium. When a cell switches to the biofilm mode of growth, it undergoes a phenotypic shift in behavior in which large suites of genes are differentially regulated.

In some embodiments, said fungi is candida. In other embodiments said biofilm is a cohort of microorganisms comprising candida.

Diseases and conditions (such as for example infections) associated with the biofilm growth usually are challenging to eradicate. It is mostly due to the fact that mature biofilms display tolerance towards antimicrobial agents and the immune response. As such, biofilms formation causes extreme problems in various situations.

For example, in the biomedical devices industry, biofilms are the main cause of infection since they are often formed on the inert surfaces of implanted and indwelling devices such as catheters, prosthetic cardiac valves, implants, artificial and intrauterine devices. No matter the sophistication, microbial infections can develop on all medical devices and tissue engineering constructs. 60-70% of nosocomial or hospital acquired infections are associated with the implantation of a biomedical device. This leads to 2 million cases annually in the U.S., costing the healthcare system over $5 billion in additional healthcare expenses.

In some cases, biofilms can be formed on the teeth of most human/animals as dental plaque, where they may cause tooth decay and gum diseases. In addition to root canal infections, ulcerations, enamel discoloring, tooth hypersensitivity, candidiasis, and so forth.

The formation of biofilms is also problematic in several food industries due to the ability to form on plants and during industrial processes. Microbes can survive long periods of time in water, animal manure, and soil, causing biofilm formation on plants or in the processing equipment. The buildup of biofilms can affect the heat flow across a surface and increase surface corrosion and frictional resistance of fluids. These can lead to a loss of energy in a system and overall loss of products. Along with economic problems biofilm formation on food poses a health risk to consumers due to the ability to make the food more resistant to disinfectants.

In produce, microorganisms attach to the surfaces and biofilms develop internally. During the washing process, biofilms resist sanitization and allow the microbes to spread across the produce. This problem is also found in ready to eat foods because the foods go through limited cleaning procedures before consumption. Due to the perishability of dairy products and limitations in cleaning procedures, resulting in the buildup of bacteria, dairy is susceptible to biofilm formation and contamination. The microbes can spoil fresh, cool and frozen products readily and contaminated products pose a health risk to consumers. Large amounts of salmonella contamination can be found in the poultry processing industry as about 50% of salmonella strains can produce biofilms on poultry farms. Salmonella increases the risk of foodborne illnesses when the poultry products are not cleaned and cooked correctly. Salmonella is also found in the seafood industry where biofilms form from seafood borne pathogens on the seafood itself as well as in water.

In shellfish and algae farms, biofouling species tend to block nets and cages and ultimately outcompete the farmed species for space and food. Microbial biofilms start the colonization process by creating microenvironments that are more favorable for biofouling species. In the marine environment, biofilms could reduce the hydrodynamic efficiency of ships and propellers, lead to pipeline blockage and sensor malfunction, and increase the weight of appliances deployed in seawater. Biofilm can also be a reservoir for potentially pathogenic bacteria in freshwater aquaculture. Additionally, formation and existence of biofilm affects the flow in desalinization fresh water pipes, recycled water pipelines and filters and pumps.

Within the biofilm ecosystem, microorganisms are less susceptible to antibacterial agents, and are better protected from the host defense system. It is also conceivable that microorganisms in the biofilm exhibit different phenotypic and genotypic characteristics than do planktonic microorganisms. Thus, treatment of such biofilm is critical when trying to maintain microbial free environment of sensitive surfaces as mentioned above. When referring to the treatment of any disease, condition or symptom caused by or associated with the formation of biofilm it should be understood to include any reduction, inhibition, amelioration or elimination of disease, condition or symptom that is related to the formation of said biofilm in any environment (including living or non-living surfaces, surfaces of medically sensitive items, natural or non-natural soft surfaces and so forth).

In another aspect the invention provides a composition comprising at least one cannabinoid compound, for use in the inhibition of the formation and/or growth of biofilm. Under such embodiments, treatment with the composition of the invention prevents the formation of biofilm.

In some embodiments, said biofilm is formed by at least one of microbe (microbial biofilm), bacteria (bacterial biofilm), protozoa (protozoal biofilm) and fungi (fungal biofilm) and (polymicronial, inter-kingdom biofilm).

In some embodiments of the present invention the term bacterial infection is drug resistant bacterial infection.

In other embodiments, said biofilm is resistant to at least one of anti-microbial, anti-fungal or anti-biotic agents.

Drug resistant bacteria includes, but not limited to any bacteria and other microorganisms that is resist to the effects of one or more drug agent such as for example an antibiotic.

Anti-microbial resistance is displayed by the ability of a microbe (bacteria, fungi, virus and so forth) to resist the effects of medication previously used to treat them, in some cases such microbes and their biofilm are multi-drug resistance. This broader term also covers anti-biotic resistance, which applies to bacteria and antibiotics. Resistant microbial biofilms are increasingly difficult to treat, and in the part typically required the use of alternative medications or higher doses, both of which were shown to be more expensive and/or more toxic. Anti-microbial resistance includes within its scope also fungi develop antifungal resistance, protozoa develop antiprotozoal resistance, and bacteria develop antibiotic resistance.

This resistance is multifactorial and complex, involving: (i) limited drug penetration into the biofilm due to the high density of extracellular matrix, (ii) drug absorption or binding by the biofilm extracellular matrix, (iii) decreased growth rate, (iv) overexpression of genes involved in drug resistance, particularly those encoding efflux pumps, (v) and multidrug tolerance due to persistent cells The outcome of immobilized microbes in biofilm in terms of pathogenicity and drug resistance emphasizes the need for new antibiofilm agents that can inhibit biofilm formation or destroy preformed biofilm without affecting microbial viability.

In some other embodiments, said condition caused by formation of biofilm is an infection. In some other embodiments, said infection is a nosocomial infection (hospital acquired infection). In other embodiments, said infection is an ear infection. In further embodiments, said infection is a dermatological infection. In further embodiments, said infection is a vaginal infection. In further embodiments, said infection is a soft tissue infection (including any type of skin membrane, mucosal membrane, vaginal membrane, rectal membrane, respiratory tract tissue, including nasal, lung, trachea, bronchi and so forth).

In further embodiments, said disease or condition caused by formation of biofilm is a is fungal infection.

In other embodiments, said disease or condition caused by formation of biofilm is a surface condition. When referring to a “surface condition” it should be understood to relate to any disease or condition that is caused by the formation of biofilm on a surface including any type of living or non-living surface such as for example liquid surfaces, any type of solid surfaces, biological surfaces such as skin and mucosal tissue, natural or non-natural soft surfaces.

In some other embodiments, a composition of the invention further comprises at least one additional agent. In some embodiments, said additional agent is a pharmaceutically active agent.

In some embodiments, said at least one additional agent is selected from an anti-fungal agent, an anti-microbial agent, an anti-bacterial agent, an anti-biotic agent, an anti-viral agent and any combinations thereof.

In other embodiments, said at least one additional agent is an agent that said disease or condition is resistant to when administered alone. Thus, under such embodiments, a disease or condition that is caused by the formation of biofilm is resistant when treated with said additional agent alone. In some cases, said disease or condition shows anti-microbial resistance towards said additional agent.

In other embodiments, said at least one additional agent is an anti-fungal agent selected from fluconazole, ketoconazole, nystatin, amphotericin B, clotrimazole, caspofungin and any combinations thereof.

In further embodiments, said at least one additional agent is an anti-biotic agent selected from penicillin family, tetracycline family, cephalosporin family, fluoroquinolones family, carbapenem family, aminoglycosides family, macrolides family, vancomycin, rifampin, doxycycline, linezolid, tetracycline, trimethoprim and any combinations thereof.

In further embodiments, said at least one additional agent is an anti-fungal agent selected from fluconazole, nystatin, amphotericin B ketoconazole, nystatin, amphotericin B, clotrimazole, caspofungin and any combinations thereof.

In further embodiments, said at least one additional agent is an anti-septic agent selected from proteins family, enzymes, charged amines family, peroxide family, iodine, and any combinations therefore.

In further embodiments, said at least one additional agent is an plant extract, such as for example polyphenols, licorice.

In another one of its aspects, the invention provides a composition comprising ARAS and any derivative thereof, for use in the reduction and/or inhibition of at least one condition selected from microbial growth, bacterial growth, fungal growth, biofilm formation, biofilm distribution, biofilm maturation, quorum sensing cascade and any combinations thereof.

The invention thus provides a composition comprising ARAS and any derivative thereof for use in the treatment of a disease, condition or symptom caused by, or associated with at least one of microbial growth, bacterial growth, fungal growth, biofilm formation, biofilm distribution, biofilm maturation, quorum sensing cascade and any combinations thereof.

Under such aspects, the invention provides a composition comprising ARAS for use in the treatment of microbial infection, bacterial infection, fungal infection and any combinations thereof.

In some embodiments, said at least one disease or condition is drug resistance. In some embodiments, said at least one disease or condition shows anti-microbial resistance.

In another one of its aspects, the invention provides a composition comprising at least one cannabinoid compound and at least one agent selected from an antimicrobial agent, an antifungal agent, an antibacterial agent an antibiotic agent.

In another one of its aspects, the invention provides a composition comprising at least one cannabinoid compound and at least one agent selected from an antimicrobial agent, an antifungal agent, an antibacterial agent, antibiotic agent for use in the treatment of a disease, condition or symptom associated with microbial infection, bacterial infection, fungal infection, cystic fibrosis, lung infections, nose and throat infections, skin infections, tissue infections, eye infections, tooth and gum infections, polyp infection, ear infection, gland infection, nail infection, feet infection, athlete foot infection, genitalia infection or any combinations thereof.

In some embodiments, said microbial infection, bacterial infection, fungal infection or any combination thereof is drug resistant. In other embodiments, said microbial infection, bacterial infection, fungal infection or any combination thereof is resistant to said at least one agent.

The invention further provides a method of treating at least one surface condition selected from microbial growth, fungal growth, biofilm formation, bacterial growth, biofilm maturation, quorum sensing cascade and any combinations thereof, said method comprises treating said surface with a composition comprising at least one cannabinoid compound and at least one agent selected from an antimicrobial agent, an antifungal agent, an antibacterial agent and any combination thereof.

In another aspect the invention provides a method of sensitizing and/or preventing biofilm formation on a surface, comprising contacting said surface with a composition comprising at least one cannabinoid compound. When referring to “sensitizing biofilm formation on a surface” should be understood as a method by which biofilm formation on said surface is diminished, inhibited or slowed down to the degree of inhibition.

The invention further provides a method of preventing the formation of biofilm on a surface, comprising contacting said surface with a composition comprising at least one cannabinoid compound.

When referring to “contacting of said surface” it should be understood to relate to applying said composition on said surface in any form, formulation or procedure known in the art, such that the at least a part of said surface is in direct interaction with said composition. In some embodiments, said contacting said surface with a composition is performed prior to, after and/or concurrent to contacting said surface (the same or approximate to the surface defined hereinabove) with at least one of antimicrobial agent, an antifungal agent, an antibacterial agent, and any combinations thereof.

The invention provides a method of treatment, prevention or inhibition of the formation or growth of at least one of fungi, fungal biofilm and any combinations thereof in a food product comprising exposing said food product to a composition comprising at least one cannabinoid compound. When referring to a “food product” it should be understood to include any substance consumed by an organism (including a mammal), to provide nutritional support for said organism. Said food product can be of plant or animal origin. Said product can be solid, liquid or semi-solid. Said product can be exposed to said composition of the invention either during storage, prior to consumption, or upon its preparation for storage or consumption. Exposure of said food product may be performed by mixing, adding, covering, dissolving, emulsifying, layering, micro-phasing, evaporating, baking, cooking, boiling, refrigerating, cooling, freezing, sublimating and any combinations thereof with a composition of the invention.

The invention further provides a method of treatment, prevention or inhibition of the formation or growth of at least one of fungi, fungal biofilm and any combinations thereof in soil or plant comprising exposing said soil or plant to a composition comprising at least one cannabinoid compound. When referring to “soil” or “plant” (including seed and/or seedling) it should be understood that the reference is to the agricultural terms relating to a soil patch used for growing plants. Exposure of said soil and/or plant to a composition of the invention includes the exposure of soil prior to the planting of a seed or a plant therein, exposure of the soil after the planting of a seed or a plant therein, exposure of the soil during the planting of a seed or a plant therein, exposure of the soil during the growth of a seed or a plant therein, exposure of the plant the planting of a seed or a plant therein. Exposure of said soil or plant includes spraying, irrigating with, spreading, mixing, adding and any combinations thereof.

The present invention relates to pharmaceutical compositions comprising at least one cannabinoid with or without a further active agent, in admixture with pharmaceutically acceptable auxiliaries, and optionally other therapeutic agents. The auxiliaries must be “acceptable” in the sense of being compatible with the other ingredients of the composition and not deleterious to the recipients thereof. In cases where the composition disclosed in this invention includes more than one active agent (for example one cannabinoid and one additional active agent such as antifungal agent, antimicrobial agent and/or antibacterial agent), said composition may be a single composition comprising both agents, or a separate compositions each comprising at least one active agent, which are administered concomitantly, separately, concurrently, parallel, simultaneously, to the same or different surface areas to be treated. The administration method is defined in the instructions for use.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrates, lubricants, colorants, flavoring agents, anti-oxidants, and wetting agents.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows the inhibition of biofilm formation of C. albicans of HU210.

FIGS. 2A-2D show HU210 effect on fungal morphology in biofilm.

FIGS. 3A-3F HU210 reduction of viable fungal cells within biofilm.

FIG. 4 shows the inhibition effect of HU210 of co-species C. albicans-S. mutans biofilm formation.

FIG. 5 shows the inhibition effect of ARAS on single and co-species biofilm formation.

FIG. 6 shows the Relative Bioluminescence Unit (RLU) of different mutant strains of bacteria V. harveyi when exposed to different sub-MIC concentrations of AEA. RLU is represented as area under the curve (AUC) and shown in relevance with the control experiment where AEA is absent. * P<0.05 (n=3).

FIG. 7 shows the endocannabinoids inhibition of S. mutans biofilm formation.

FIG. 8 shows the 2-AG (endocannabinoid) dose-dependent inhibition of C. albicans biofilm formation.

FIG. 9 shows the AEA (endocannabinoid) dose-dependent inhibition of C. albicans biofilm formation.

FIGS. 10A-10D show the CSLM of S. mutans biofilm—The live bacteria are marked in green and the dead bacteria are marked in red. The AEA show a dose-dependent inhibition of S. mutans biofilm formation.

FIGS. 11A-11D show CLSM images of treated biofilms of P. aeruginosa. Effect of AEA PEA (endocannabinoids/endocannabinoids derivatives) on biofilm of P. aeruginosa. Both treatments resulted in reduced layers/depth of biofilm.

FIGS. 12A-12E show the effect of AEA and AraS on eradication of formed biofilm on MRSA 33592 (12A=control; 12B, 12C=AEA, 12D, 12E=AraS)

FIGS. 13A-13I show the effect of ECs on spreading ability of MRSA. All tested MRSA strains demonstrated strong ability to spread on the agar (control 13A, 13D, 13G). Both ECs, AEA and in less impact ARAS were able to reduce colony spreading. AEA at 64 μg/ml reduced diameter of the colony of CI, 33592 and 43000 strains by 88% (13B), 84% (14E), and 73% (13H), respectively, as compared to untreated controls (13A, 13D, 13G). ARAS at sub-MICs was able to inhibit colony spreading of CI, 33592 and 43000 strains by 64% (13C), 65% (13F), and 46% (13I), respectively, as compared to untreated controls (13A, 13D, 13G).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Example 1: Anti-Biofilm Effect of Synthetic Cannabinoid HU210

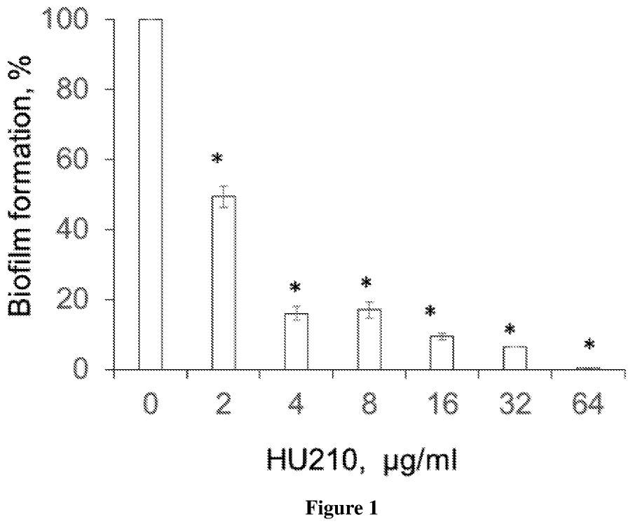

FIG. 1 demonstrated pronounced dose-dependent inhibitory effect of HU210 C. albicans biofilm formation. Minimal biofilm inhibitory concentration 50 (50% of biofilm inhibition) MBIC50 was recorded already at lowest tested dose of HU210=2 μg/ml (FIG. 1). Almost no biofilm formed at highest tested dose of HU210=64 μg/ml (FIG. 1). In contrast to the strong anti-biofilm activity of HU210, no effect on fungal growth was detected, since minimal inhibitory concentration (MIC) of HU210 was not detected at tested doses.

### Example 2: HU210 Affects Fungal Morphology in Biofilm

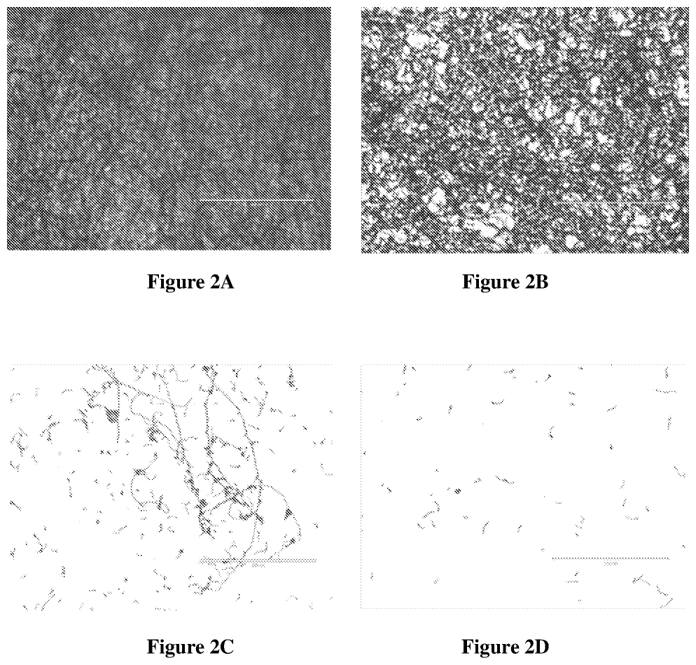

Microscopic observation showed that HU210 dramatically alters biofilm morphologic composition. As shown in FIG. 2, untreated control biofilm (FIG. 2A) consisted of candidal branched hyphae and characterized by highly dense mycelium. However, HU210 already at 8 μg/ml influenced fungal morphology (FIG. 2C). In addition, density of fungal mycelium decreased dose-dependently (FIG. 2B-D). Furthermore, HU210 at dose of 64 μg/ml lead to the alteration of yeast-to-hyphae transition resulting in the appearance of mainly yeast form of C. albicans (FIG. 2D).

### Example 3: HU210 Reduces Viable Fungal Cells within Biofilm

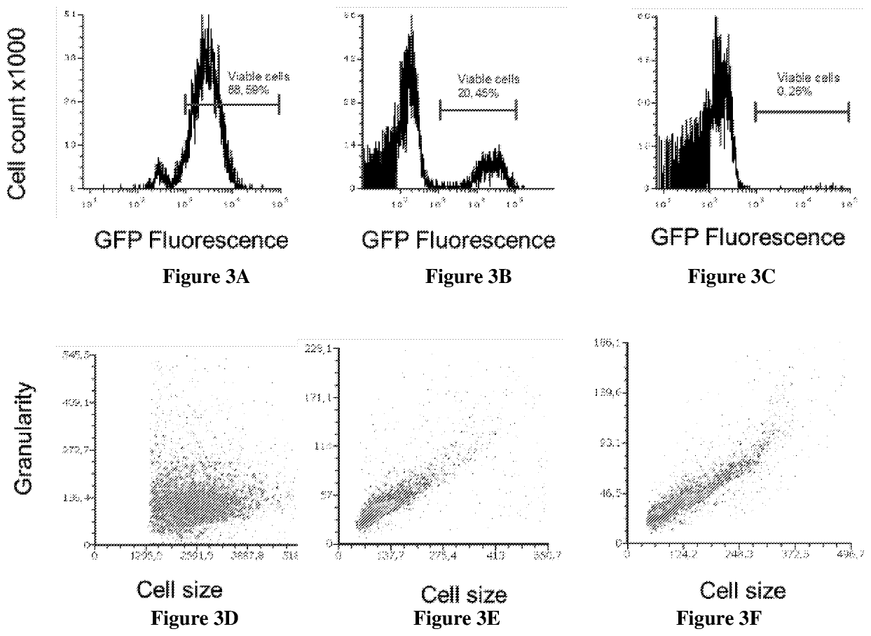

Flow cytometry analysis demonstrated dramatic decrease of viable cells in biofilm due to exposure to HU210 (FIG. 3). Pronounced reduction of viable C. albicans cells from 88% in untreated control (FIG. 3A) to 20% in biofilm treated with 8 μg/ml of HU210 (FIG. 3B) was detected. Finally, highest tested dose of HU210=64 μg/ml totally reduced viable cells in fungal biofilm (FIG. 3C). Furthermore, granularity and cell size, which reflect mycelium density and morphologic form, respectively were altered by HU210. Granularity was reduced from 136 AU in control (FIG. 3D) to 50 AU and 40 AU in samples treated with 8 μg/ml (FIG. 3E) and 64 μg/ml (FIG. 3F), respectively. Cell size was reduced from 260 AU in control (FIG. 3D) to 110 AU and 100 AU in samples treated with 8 μg/ml (FIG. 3E) and 64 μg/ml (FIG. 3F), respectively. Flow cytometry results obviously support morphologic observation.

### Example 4: HU210 Inhibits Co-Species C. albicans-S. mutans Biofilm Formation

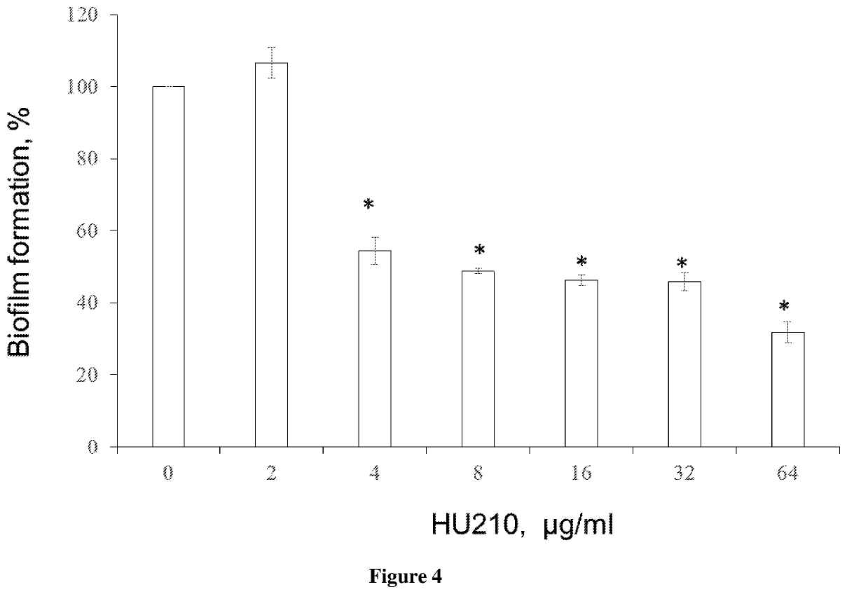

FIG. 4 demonstrated inhibitory effect of HU210 on mixed C. albicans-S. mutans biofilm formation. MBIC50 was recorded already at 4 μg/ml of HU210. Growth of co-culture was not affected by HU210 at all tested doses of HU210. In contrast, HU210 exhibited pronounced inhibitory effect (MIC=2 μg/ml) towards single S. mutans specie growth. No streptococcal biofilm was formed at this concentration of HU210 (data not shown).

### Example 5: Antimicrobial Activity of Selected Endocannabinoids

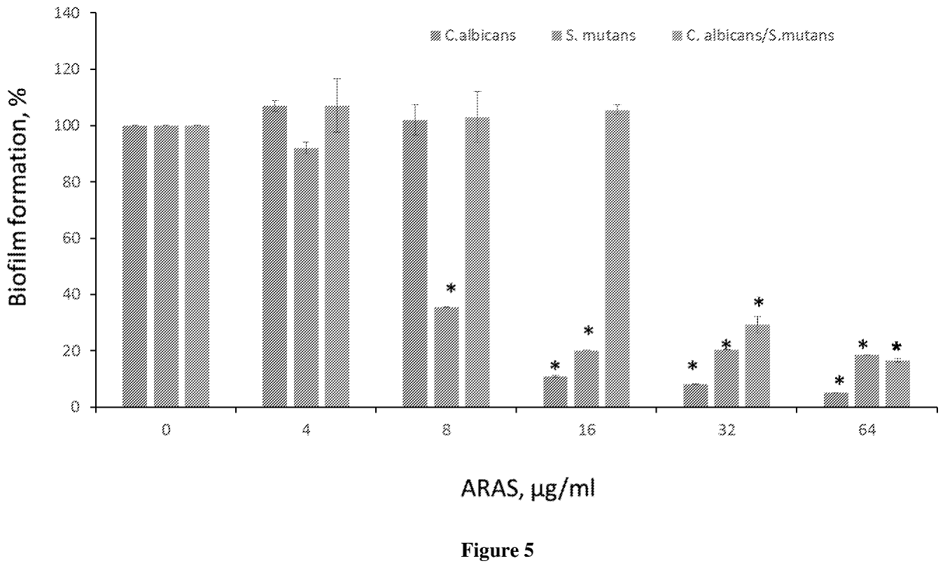

FIG. 5 demonstrated dose-dependent inhibitory effect of ARAS on S. mutans, C. albicans and mixed S. mutans-C. albicans biofilm formation. ARAS at dose of 8 μg/ml was able to inhibit single S. mutans biofilm formation by more than 50%. MBIC50 for single C. albicans and mixed S. mutans-C. albicans biofilms was detected at 16 μg/ml and 32 μg/ml of ARAS, respectively. In contrast growth of S. mutans was inhibited only at highest tested dose of ARAS (MIC=64 μg/ml), while single C. albicans and mixed S. mutans-C. albicans growth was not affected at all tested concentrations of ARAS (MIC>64 μg/ml) (data not shown).

### Example 6: Antimicrobial Activity Against Resistance Microbes (Bacteria-Fungal)

TABLE 1
Effect of combination of AEA and methicillin against methicillin resistant staphylococci
S. aureus MRSA 24433
Growth, μg/ml

| MIC AEA | MIC METH | FIC AEA | FIC METH | FICI | effect |
|---|---|---|---|---|---|
| >64 | >64 | 16 | 16 | <0.5 | synergy |

Biofilm, μg/ml

| MBIC AEA | MBIC METH | FBIC AEA | FBIC METH | FBICI | effect |
|---|---|---|---|---|---|
| 32 | >64 | 8 | 16 | <0.5 | synergy |

As shown in Table 1, AEA in combination with methicillin has synergistic effect either on growth or on biofilm formation of methicillin resistant staphylococci. Both agent have no effect on bacterial growth (MIC>64 μg/ml), while in combination MIC of each compound in combination decreased by more than 4-fold. Calculated FICI is less than 0.5 which indicates on synergistic activity between these agents towards bacterial growth. Similar results were obtained concerning biofilm formation. MBIC of each compound in combination was less than MBIC of appropriate compound alone by 4 fold or more. Calculated FBICI is less than 0.5, which indicates on synergistic effect between these agents towards biofilm formation.

### Example 7: Anti Quorum Sensing Effect

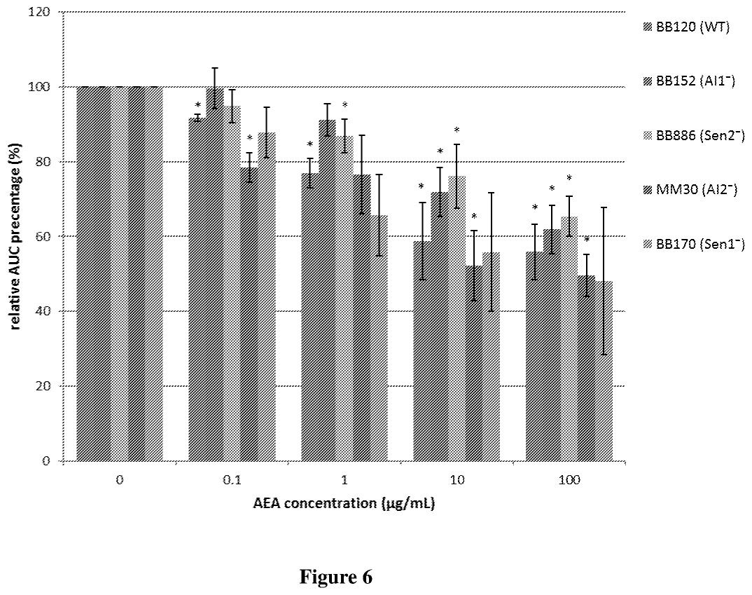

FIG. 6 Relative Bioluminescence Unit (LUM/(O.D(595 nm))) (RLU) of different mutant strains of bacteria V. harveyi when exposed to different sub-MIC concentrations of AEA. RLU is represented as area under the curve (AUC) and shown in relevance with the control experiment where AEA is absent. * P<0.05 (n=3).

The quorum sensing assays indicate on an inhibition in the presence of AEA. A dose response is observed up to the 100 μg/ml. A dose-response in quorum sensing was observed up to 100 mg/ml AEA, which are concentrations below the MIC.

Selected cannabinoids demonstrated specific non-killing anti-biofilm effect towards bacterial and fungal pathogens. Moreover, selected cannabinoid, AEA, exhibited effect in combination with antibiotic, towards bacteria that is resistant to this antibiotic. Thus, tested cannabinoids could be promising therapeutics against biofilm-associated infections. Furthermore, they could be administrated together with antibiotics in order to: 1. affect resistant bacteria; 2. reduce antibiotic-associated adverse effects.

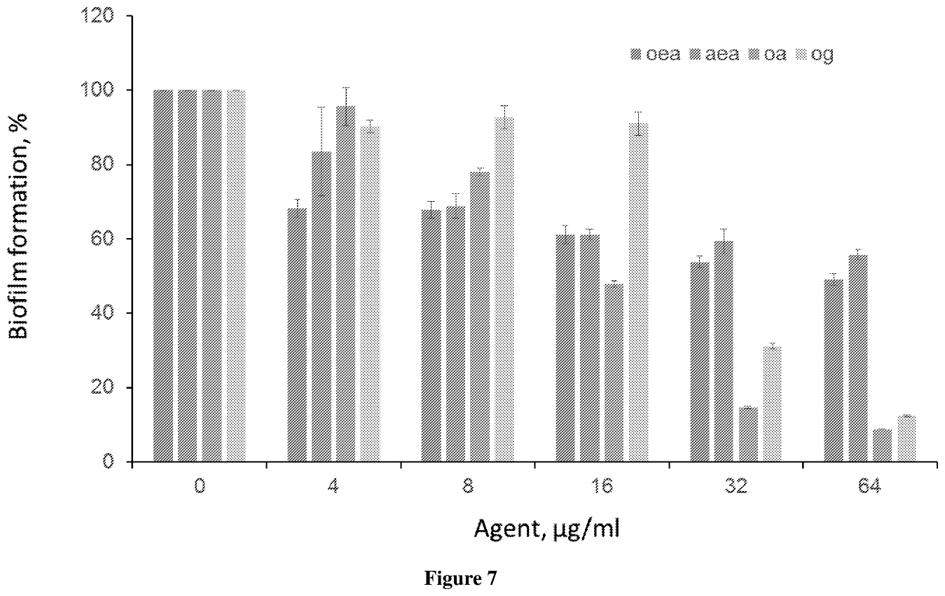

FIG. 7 demonstrated dose-dependent inhibitory effect of OEA, AEA, OA and OG on S. mutans biofilm formation. Agents OA, OG and OEA exhibited MBIC50 at 16, 32 and 64 μg/ml, respectively. AEA was less effective, however also showed inhibition of S. mutans biofilm formation by 45% at highest tested dose of 64 μg/ml. Bacterial growth was not affected by any of the tested agents at all tested doses (MIC>64 μg/ml).

### Example 8: Effect of Combination of Endocannabinoids with Antibiotics/Antimycotic Agents on Resistant Bacteria/Fungi Growth and Biofilm Formation

### Abbreviations and Explanations:

| | |
|---|---|
| MIC AEA/ARAS-MIC | AEA/ARAS alone |
| MIC METH/AMP/GEN/FLU-MIC | methicillin/ampicillin/gentamycin/fluconazole alone |
| FIC AEA/ARAS - MIC | AEA/ARAS in combination with |
| | methicillin/ampicillin/gentamycin/fluconazole |
| FIC METH/AMP/GEN/FLU - MIC | methicillin/ampicillin/gentamycin/fluconazole in |
| | combination with AEA/ARAS |
| FICI | fractional inhibitory concentration index |
| MBIC AEA/ARAS-MBIC | AEA/ARAS alone |
| MBIC METH/AMP/GEN/FLU-MBIC | methicillin/ampicillin/gentamycin/fluconazole alone |
| FBIC AEA/ARAS - MBIC | AEA/ARAS in combination with |
| | methicillin/ampicillin/gentamycin/fluconazole |
| FBIC METH/AMP/GEN/FLU - MBIC | methicillin/ampicillin/gentamycin/fluconazole in |
| | combination with AEA/ARAS |
| FBICI | fractional biofilm inhibitory concentration index |
| Synergistic effect* | FICI/FBIC of <0.5 |
| Partial synergism* | 0.5 > FICI/FBIC < 1 |
| Additive effect* | FICI/FBIC = 1 |
| Indifference* | 1 > FICI/FBIC < 4 |
| Antagonism* | FICI/FBIC of more than 4 |

(*) Lee WX, Basri DF, Ghazali AR Bactericidal Effect of Pterostilbene Alone and in Combination with Gentamicin against Human Pathogenic Bacteria. Molecules. 2017 Mar 17;22(3))

Effect of Combination of ARAS with Fluconazole Against Fluconazole Resistant C. albicans Strains

Table 2 demonstrated that each agent alone was non-effective against biofilm formation of both resistant fungal strains (MBIC 64 μg/ml or >64 μg/ml). However, combination of these agents reduced MBIC of ARAS by 2-fold, while MBIC of fluconazole was reduced by more than 32- and 16-fold. Thus, this combination was defined as partial synergistic towards biofilm formation of both tested fluconazole resistant C. albicans strains. Growth of these fungal strains was not affected either by each agent alone or in combination (data not shown).

Effect of Combination of ARAS with Different Antibiotics Against Methicillin Resistant Staphylococcus aureus (MRSA) Strains

TABLE 2
Effect of combination of ARAS with fluconazole against
fluconazole resistant C. albicans strains

| Biofilm | | | | | | |
|---|---|---|---|---|---|---|
| | MBIC | MBIC | FBIC | FBIC | | |
| strain | ARAS | FLU | ARAS | FLU | FBICI | Effect |
| DSY551 | 64 | >64 | 32 | 2 | >0.5 < 1 | partial |
| | | | | | | synergism |
| DSY735 | 64 | >64 | 32 | 4 | >0.5 < 1 | partial |
| | | | | | | synergism |

TABLE 3
Effect of combination of ARAS and methicillin against MRSA strains
A
S.aureus MRSA 33592
Growth

| MIC ARAS | MIC METH | FIC ARAS | FIC METH | FICI | effect |
|---|---|---|---|---|---|
| 64 | 32 | 16 | 8 | <0.5 | synergy |

Biofilm

| MBIC | MBIC | FBIC | FBIC | | |
|---|---|---|---|---|---|
| ARAS | METH | ARAS | METH | FBICI | effect |
| 32 | 32 | 16 | 8 | >0.5 < 1 | partial synergy |

B
S.aureus MRSA 24433
Growth

| MIC ARAS | MIC METH | FIC ARAS | FIC METH | FICI | effect |
|---|---|---|---|---|---|
| >256 | >64 | >64 | >64 | >1 < 4 | indifferent |

Biofilm

| MBIC ARAS | MBIC METH | FBIC ARAS | FBIC METH | FBICI | effect |
|---|---|---|---|---|---|
| 32 | >64 | 16 | 16 | >0.5 < 1 | additive |

C
S.aureus MRSA 43300
Growth

| MIC ARAS | MIC METH | FIC ARAS | FIC METH | FICI | effect |
|---|---|---|---|---|---|
| 64 | 32 | 16 | 2 | <0.5 | synergy |

Biofilm

| MBIC ARAS | MBIC METH | FBIC ARAS | FBIC METH | FBICI | effect |
|---|---|---|---|---|---|
| 32 | 32 | 8 | 8 | <0.5 | synergy |

TABLE 4
Effect of combination of ARAS and gentamycin against MRSA strain
S.aureus MRSA 33592
Growth

| MIC ARAS | MIC GEN | FIC ARAS | FIC GEN | FICI | effect |
|---|---|---|---|---|---|
| 32 | 128 | 4 | 4 | <0.5 | synergy |

Biofilm

| MBIC ARAS | MBIC GEN | FBIC ARAS | FBIC GEN | FBICI | |
|---|---|---|---|---|---|
| 32 | 128 | 4 | 4 | <0.5 | synergy |

TABLE 5
Effect of combination of ARAS and ampicillin against MRSA strains
A
S.aureus MRSA 33592
Growth

| MIC ARAS | MIC AMP | FIC ARAS | FIC AMP | FICI | effect |
|---|---|---|---|---|---|
| 32 | 128 | 8 | 64 | <0.5 < 1 | partial synergy |

Biofilm

| MBIC ARAS | MBIC AMP | FBIC ARAS | FBIC AMP | FBICI | effect |
|---|---|---|---|---|---|
| 32 | 128 | 16 | 32 | <0.5 < 1 | partial synergy |

B
S.aureus MRSA 43300
Growth

| MIC ARAS | MIC AMP | FIC ARAS | FIC AMP | FICI | effect |
|---|---|---|---|---|---|
| 64 | 256 | 16 | 16 | <0.5 | synergy |

Biofilm

| MBIC ARAS | MBIC AMP | FBIC ARAS | FBIC AMP | FBICI | effect |
|---|---|---|---|---|---|
| 32 | 256 | 8 | 64 | <0.5 | synergy |

Combination of ARAS with various antibiotics was also effective against methicillin-resistant strains of S. aureus. As shown in Table 3, combination of ARAS with methicillin has synergistic effect on two methicillin-resistant strains MRSA 33592 (Table 3A) and MRSA 43300 (Table 3C) growth. This combination was also effective against biofilm formation: synergy was detected against MRSA 43300 (Table 3C) and partial synergy was detected against MRSA 33592 (Table 3A) biofilm formation. In addition, combination of ARAS with gentamicin or ampicillin exhibited synergistic (Table 4) or partial synergistic effect (Table 5A), respectively, towards MRSA 33592 growth and biofilm formation. Furthermore, combination of ARAS with ampicillin demonstrated synergistic effect against MRSA 43300 growth and biofilm formation (Table 5B).

Effect of Combination of AEA with Different Antibiotics Against MRSA Strains.

TABLE 6
Effect of combination of AEA and methicillin against MRSA strains.
A
S.aureus MRSA 33592
Growth

| MIC AEA | MIC METH | FIC AEA | FIC METH | FICI | effect |
|---|---|---|---|---|---|
| >256 | 32 | 16 | 16 | <0.5 | synergy |

Biofilm

| MBIC AEA | MBIC METH | FBIC AEA | FBIC METH | FBICI | effect |
|---|---|---|---|---|---|
| 64 | 32 | 8 | 8 | <0.5 | synergy |

B
S.aureus MRSA 24433
Growth

| MIC AEA | MIC METH | FIC AEA | FIC METH | FICI | effect |
|---|---|---|---|---|---|
| >256 | >64 | 16 | 16 | <0.5 | synergy |

Biofilm

| MBIC AEA | MBIC METH | FBIC AEA | FBIC METH | FBICI | effect |
|---|---|---|---|---|---|
| 32 | >64 | 8 | 16 | <0.5 | synergy |

C
S.aureus MRSA 43300
Growth

| MIC AEA | MIC METH | FIC AEA | FIC METH | FICI | effect |
|---|---|---|---|---|---|
| >256 | 32 | 16 | 8 | <0.5 | synergy |

Biofilm

| MBIC AEA | MBIC METH | FBIC AEA | FBIC METH | FBICI | effect |
|---|---|---|---|---|---|
| >256 | 32 | 32 | 8 | <0.5 | synergy |

TABLE 7
Effect of combination of AEA and gentamicin against MRSA strain.
S.aureus MRSA 33592
Growth

| MIC AEA | MIC GEN | FIC AEA | FIC GEN | FICI | effect |
|---|---|---|---|---|---|
| >256 | 128 | 8 | 4 | <0.5 | synergy |

Biofilm

| MBIC AEA | MBIC GEN | FBIC AEA | FBIC GEN | FBICI | effect |
|---|---|---|---|---|---|
| 64 | 128 | 8 | 8 | <0.5 | synergy |

TABLE 8
Effect of combination of AEA and ampicillin against MRSA strains.
A
S.aureus MRSA 33592
Growth

| MIC AEA | MIC AMP | FIC AEA | FIC AMP | FICI | effect |
|---|---|---|---|---|---|
| >256 | 128 | 8 | 8 | <0.5 | synergy |

Biofilm

| MBIC AEA | MBIC AMP | FBIC AEA | FBIC AMP | FBICI | effect |
|---|---|---|---|---|---|
| 64 | 128 | 8 | 8 | <0.5 | synergy |

B
S.aureus MRSA 43300
Growth

| MIC AEA | MIC AMP | FIC AEA | FIC AMP | FICI | effect |
|---|---|---|---|---|---|
| >256 | >128 | 16 | 8 | <0.5 | synergy |

Biofilm

| MBIC AEA | MBIC AMP | FBIC AEA | FBIC AMP | FBICI | effect |
|---|---|---|---|---|---|
| >256 | >128 | 16 | 8 | <0.5 | synergy |

Agent AEA demonstrated notable synergistic effect being in combination with various antibiotics against MRSA strains growth and biofilm formation. Combination of AEA with methicillin (Table 6), gentamicin (Table 7) or ampicillin (Table 8) showed strong synergistic effect against all tested MRSA strains growth and biofilm formation. The most pronounced synergistic effect was detected in combination of AEA with gentamicin against MRSA 33592 growth (Table 7). These bacteria were highly resistant to each agent alone (MIC of AEA>256, MIC of gentamicin=128). However, combination of AEA and gentamicin dramatically decreased MIC of AEA by more than 32-fold and MIC of gentamicin by 32-fold (Table 7).

Selected cannabinoids obviously demonstrated specific non-killing anti-biofilm effect towards bacterial and fungal pathogens. Moreover, selected endocannabinoids, AEA and ARAS, exhibited obvious synergistic effect in combination with various antibiotics towards methicillin-resistant strains of S. aureus. Thus, tested cannabinoids could be promising therapeutics against biofilm-associated infections. Furthermore, they could be administrated together with antibiotics in order to: 1. affect resistant bacteria; 2. reduce antibiotic-associated adverse effects.

### Example 9: Dose-Dependent Inhibition of C. albicans Biofilm Formation

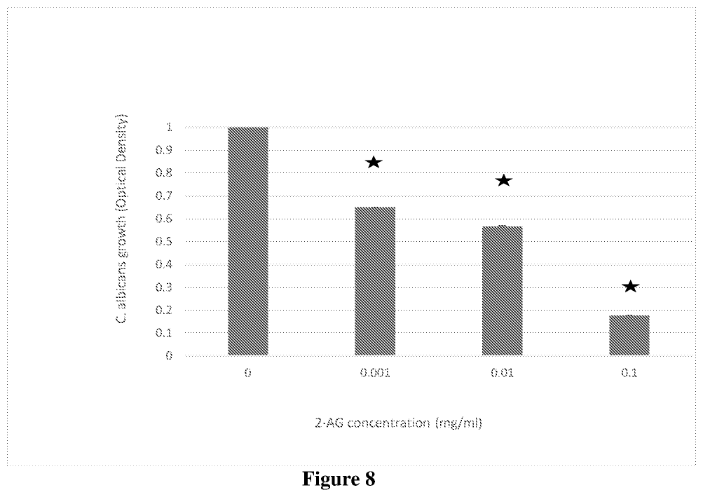

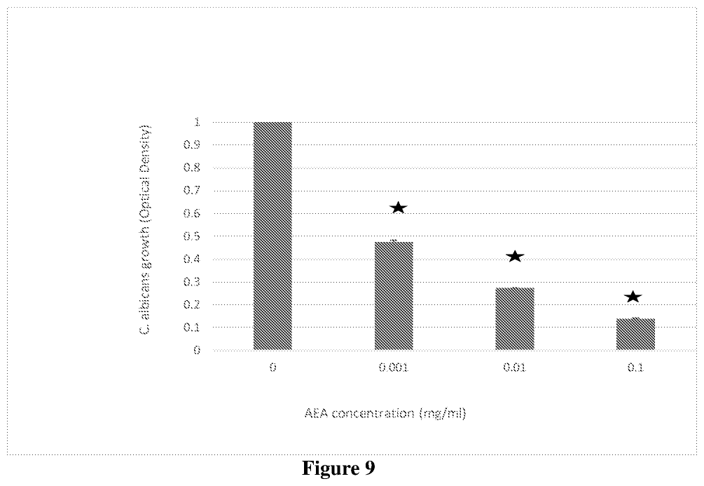

To investigate the effect of the agents on preformed biofilms, biofilms were allowed to mature in for 24 h at 37° C. in a 6-well plate. The biofilms were washed twice with PBS. The active agents were then applied. The plates were further incubated for 24 h at 37° C. The amounts of biofilms, were determined quantitatively using a standard MTT reduction assay as described previously. Briefly, biofilms were overlaid with 100 mM of 3-(4,5-dimethyl-2-thiazolyl)-2,5-diphenyl-2H-tetrazolium bromide (MTT) and incubated for 2 h at 37° C. Under these conditions, the lightly yellowish MTT was reduced to a blue tetrazolium salt accumulated within the metabolic active biofilms. The stain was then dissolved in DMSO and the absorbance value was measured at 570 nm. The accumulation of tetrazolium salt by the reduction of MTT is proportional to the number of viable cells growing in biofilm. Prior to dissolving in DMSO, biofilms were photographed and visualized. Assay was performed in triplicate. FIG. 8 and FIG. 9 show the MTT assay of C. albicans biofilm wherein the endocannabinoids (AEA/2-AG) show a dose-dependent inhibition of C. albicans biofilm formation.

### Example 10

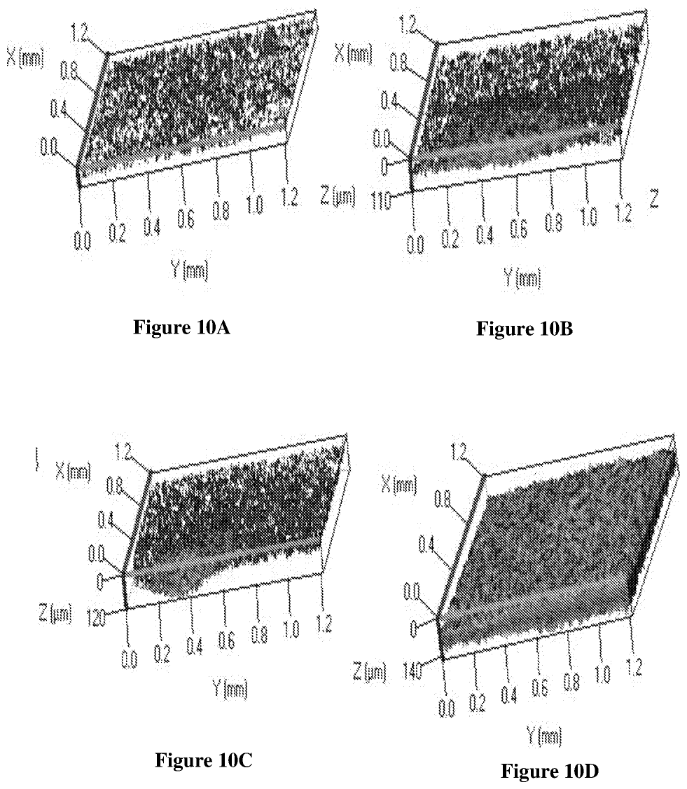

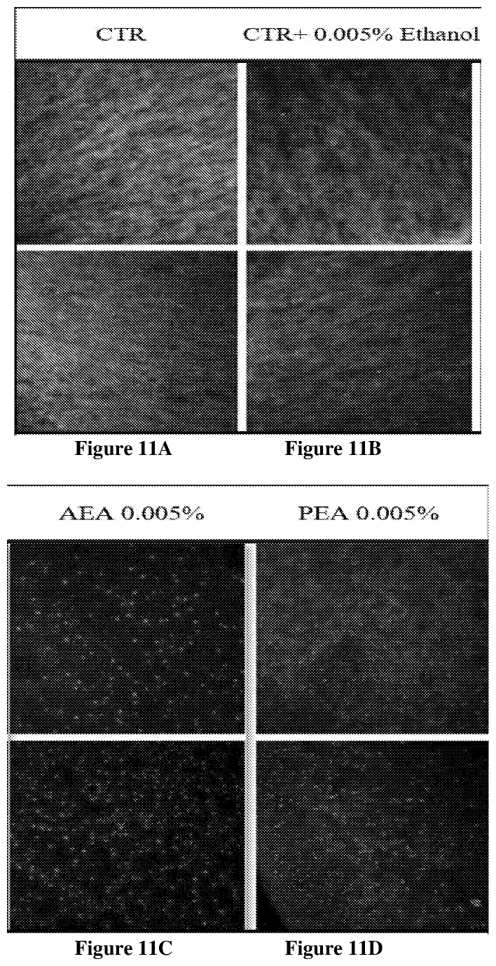

The bacterial viability and vitality of was analyzed by CLSM (Olympus Fluoview 300, Olympus, Japan) with a UPLSA 10×/0.4 lenses. The biofilm samples were grown overnight on 96 well. The biofilm was washed carefully using 200 μl PBS solution after overnight incubation, and then stained with 50 μl of LIVE/DEAD BacLight fluorescent dye (Invitrogen Life Technologies, Carlsbad, Calif., USA) (1:100) for 20 min in the dark, at room temperature. This staining allowed to distinguish the live organisms from the dead ones. Living bacteria were stained with SYTO 9 dye and were observed in green color while dead bacteria were stained with PI dye and were observed in red color. The biofilm thickness was examined by generating the optical sections that were acquired at spacing steps of 10 μm Image J program (The National Institute of Health) was used for fluorescence analysis which calculates the fluorescence intensity per area for each color separately. FIG. 10 shows the CSLM of S. mutans biofilm wherein the live bacteria are marked in green and the dead bacteria are marked in red. The AEA show a dose-dependent inhibition of S. mutans biofilm formation. FIG. 11 shows the effect of AEA PEA (endocannabinoids/endocannabinoids derivatives on biofilm of P. aeruginosa. Both treatments resulted in reduced layers/depth of biofilm. AEA had a more significant reduction in biofilm density.

### Example 11

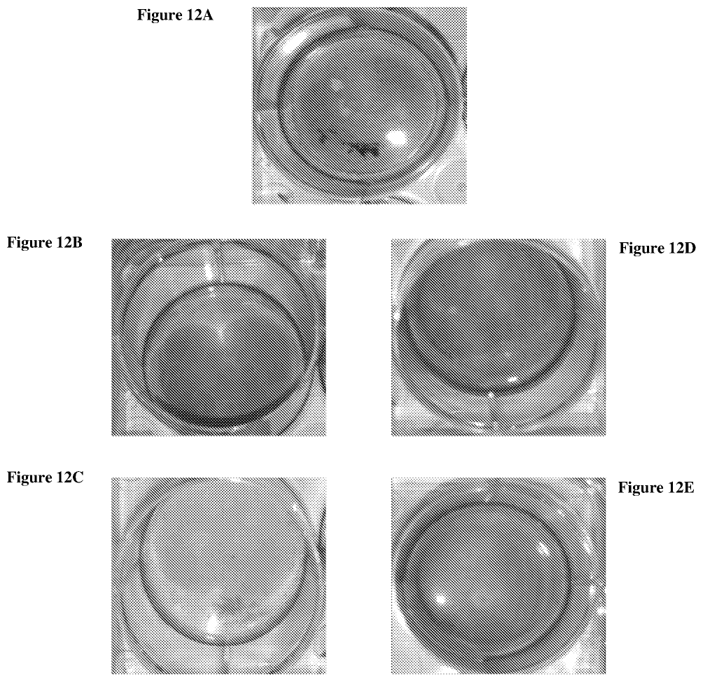

After incubation for 24 h, supernatant-fluid was removed by aspiration and the wells were carefully washed twice with phosphate-buffered saline (PBS, pH 7.4). The biofilm was measured by crystal violet staining. Briefly, 0.02% crystal violet was placed on top of the biofilm for 45 min, which were then washed twice with DDW to remove unbound dye. Figure. 12 shows the effect of AEA and AraS on eradication of formed biofilm on MRSA 33592 (12A=control, 12B, 12C=AEA, 12D, 12E=AraS).

### Example 12

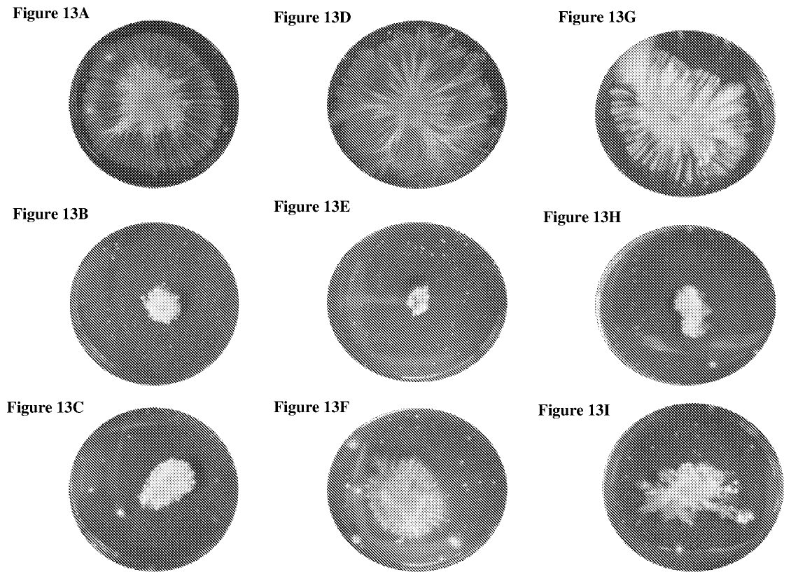

The swimming assay was performed on soft agar plates. 0.2% agar medium was prepared and autoclaved. The bacteria were exposed to the tested agents. 3 μl of overnight bacterial culture (O.D 595˜0.5) was inoculated at the centre of the agar plate. Agar plates without active agents served as controls. The plates were then incubated for 15 h. To analyze the results, the area of the motility halos was measured using Image J software (National Institute of Health) and compared with the control. FIG. 13 shows the effect of ECs on spreading ability of MRSA. All tested MRSA strains demonstrated strong ability to spread on the agar (control 13A, 13D, 13G). Both ECs, AEA and in less impact ARAS were able to reduce colony spreading. AEA at 64 μg/ml reduced diameter of the colony of CI, 33592 and 43000 strains by 88% (13B; Table 9), 84% (13E; Table 9), and 73% (13H; Table 9), respectively, as compared to untreated controls (13A, 13D, 13G). ARAS at sub-MICs was able to inhibit colony spreading of CI, 33592 and 43000 strains by 64% (13C; Table 9), 65% (13F; Table 9), and 46% (13I; Table 9), respectively, as compared to untreated controls (FIG. 13A, 13D, 13G).

TABLE 9

| | |
|---|---|
| MRSA strain | Endocannabinoid |

| | | |
|---|---|---|
| | AEA 64 μg/ml | ARAS 64 μg/ml |
| CI | 88 ± 1.9 | 64 ± 2.5 |
| | AEA 64 μg/ml | ARAS 16 μg/ml |
| 33592 | 84 ± 1.8 | 65 ± 3.4 |
| | AEA 64 μg/ml | ARAS 32 μg/ml |
| 43300 | 73 ± 2.6 | 46 ± 2.8 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-32. (canceled)

33. A method of treating at least one surface condition selected from microbial growth, fungal growth, biofilm formation, bacterial growth, biofilm maturation, quorum sensing cascade and any combinations thereof, said method comprises treating said surface with a composition comprising at least one cannabinoid compound and at least one agent selected from an antimicrobial agent, an antifungal agent, an antibacterial agent and any combination thereof.

34. A method according to claim 33, wherein said at least one cannabinoid is an endocannabinoid.

35. A method according to claim 33, wherein said at least one cannabinoid is selected from ARAS (arachidonoyl serine), 2AG (2-arachidonoyl glycerol), AEA (arachidonoyl ethanolamide), OEA (oleoyl ethanolamide), OG (oleoyl glycine), OA (oleoyl alanine), HU-210, HU-308, PEA (palmitoyl ethanolamide) HU-433, AraG (Arachidonoyl glycine), PG (Palmitoyl glycine), AraA (Arachidonoyl alanine), PA (Palmitoyl alanine), PS (Palmitoyl serine), OS (Oleoyl serine), 2-arachidonoyl glyceryl ether, 2-oleoyl glyceryl ether, 2-palmitoyl glyceryl ether and any derivative or combinations thereof.

36. A method according to claim 33, wherein said antifungal agent is selected from fluconazole, nystatin, amphotericin B, fluconazole, nystatin, amphotericin B, fluconazole, nystatin, amphotericin B, fluconazole, ketoconazole, nystatin, amphotericin B, clotrimazole, caspofungin and any combinations thereof.

37. A method according to claim 33, wherein said antibacterial agent is selected from penicillin family, cephalosporin family, fluoroquinolones family, carbapenem family, aminoglycosides family, macrolides family, vancomycin, rifampin, doxycycline, linezolid, tetracycline, trimethoprim and any combinations thereof.

38. A method according to claim 33, wherein said at least one condition is drug resistance.

39. A method according to claim 33, wherein said at least one condition is resistance to said at least one agent.

40. A method of sensitizing and/or preventing biofilm formation on a surface, comprising contacting said surface with a composition comprising at least one cannabinoid compound.

41. A method according to claim 40, wherein contacting said surface with a composition is performed prior to, after and/or concurrent to contacting said surface with at least one of antimicrobial agent, an antifungal agent, an antibacterial agent, and any combinations thereof.

42. (canceled)

43. A method of treatment, prevention or inhibition of the formation or growth of at least one of fungi, fungal biofilm and any combinations thereof in at least one of food product, soil and plant, comprising exposing said at least one of food product, soil and plant to a composition comprising at least one cannabinoid compound.

44. (canceled)
